(19) United States
(12) Patent Application Publication
(10) Pub. No.: US 2013/0028865 A1
(43) Pub. Date: Jan. 31, 2013

(54) Combination of a Macrocyclic Inhibitor of HCV, A Non-Nucleoside and a Nucleoside

(71) Applicants: Tse-I Lin, Mechelen (BE); Oliver Lenz, Sint-Katelijne-Waver (BE); Pierre Jean-Marie Bernard Raboisson, Rosieres (BE)

(72) Inventors: Tse-I Lin, Mechelen (BE); Oliver Lenz, Sint-Katelijne-Waver (BE); Pierre Jean-Marie Bernard Raboisson, Rosieres (BE)

(73) Assignees: MEDIVIR AB, Huddinge (SE); JANSSEN PHARMACEUTICALS, INC., Titusville, NJ (US)

(21) Appl. No.: 13/640,657

(22) PCT Filed: Apr. 13, 2011
(86) PCT No.: PCT/EP2011/055836

§ 371 Date: Oct. 11, 2012

(30) Foreign Application Priority Data
Apr. 13, 2010 (EP) 10159825.8

(51) Int. Cl. A61K 31/55; A61P 31/14; A61K 38/21

(52) U.S. Cl. 424/85.4; 514/214.02

(57) ABSTRACT

The present invention relates to a combination of a macrocyclic HCV protease inhibitor, a macrocyclic non-nucleoside HCV polymerase inhibitor and a nucleoside HCV polymerase inhibitor.

### FIELD OF THE INVENTION

The present invention relates to a combinations of a macrocyclic NS3/4A protease inhibitor of HCV, a HCV NS5B polymerase inhibiting non-nucleoside and a HCV NS5B polymerase inhibiting nucleoside.

### BACKGROUND OF THE INVENTION

Hepatitis C virus (HCV), a member of the Flaviviridae family of viruses in the hepacivirus genus, is the leading cause of chronic liver disease worldwide. Although the development of diagnostics and blood screening has considerably reduced the rate of new infections, HCV remains a global health burden due to its chronic nature and its potential for long-term liver damage. There are six major HCV genotypes (1-6) and multiple subtypes (represented by letters). Genotype 1b is predominant in Europe, while genotype 1a is predominant in North America. Genotype is clinically important in determining potential response to therapy and the required duration of such therapy.

HCV is mainly transmitted by blood contact. Following initial acute infection, a majority of infected individuals develops chronic hepatitis because HCV replicates preferentially in hepatocytes but is not directly cytopathic. Over decades, a considerable number of infected persons develop fibrosis, cirrhosis and hepatocellular carcinoma, with chronic HCV infection being the leading cause for liver transplantation. This and the number of patients involved, has made HCV the focus of considerable medical research.

Replication of the genome of HCV is mediated by a number of enzymes, amongst which is HCV NS3/4A serine protease and its associated cofactor, NS4A. Another essential enzyme in this process is NS5B polymerase. Both NS3/4A serine protease and NS5B polymerase are considered to be essential for viral replication and inhibitors of these enzymes are considered drug candidates for HCV treatment.

Current standard of care consists of a combination therapy of weekly pegylated interferon-α (IFN-α) and twice-daily ribavirin, and is able to cure ˜80% of patients infected by genotype 2 or 3, but only 40 to 50% of genotype 1 patients. Apart from the low success rate in genotype 1 patients, this treatment is also associated with a range of side effects including flu-like symptoms, anemia and depression. Hence there is a need for safer and more potent drugs that in particular overcome the disadvantages of current HCV therapy such as side effects, limited efficacy, poor tolerance, the emergence of resistance, as well as compliance failures.

The high error rate of HCV polymerase together with a high viral turnover results in a heterogeneous population of HCV genomes within each patient and, depending on the frequency and fitness of these variants, provides a high hurdle for viral eradication. Thus it is likely that future therapies will consist of combinations of several antiviral drugs, if needed with IFN-α and ribavirin, to enhance the antiviral effect and also raise the threshold for resistance development, ultimately improving sustained virologic response (SVR) rates.

Various agents have been described that inhibit HCV NS3/4A serine protease. WO 05/073195 discloses linear and macrocyclic NS3 serine protease inhibitors with a central substituted proline moiety and WO 05/073216 with a central cyclopentyl moiety. Amongst these, the macrocyclic derivatives are attractive due to their potency and interesting pharmacokinetic profile. WO 2007/014926 discloses a series of macrocyclic NS3 serine protease inhibitors. Of these, the compound (1R,4R,6S,15R,17R)-cis-N-[17-[2-(4-isopropylthiazole-2-yl)-7-methoxy-8-methyl-quinolin-4-yloxy]-13-methyl-2,14-dioxo-3,13-diazatricyclo[13.3.0.0^{4,6}]octadec-7-ene-4-carbonyl](cyclopropyl)sulfonamide, which can also be referred to as (1R,4R,6S,7Z,15R,17R)-N-[17-[2-(4-isopropylthiazole-2-yl)-7-methoxy-8-methyl-quinolin-4-yloxy]-13-methyl-2,14-dioxo-3,13-diazatricyclo[13.3.0.0^{4,6}]octadec-7-ene-4-carbonyl](cyclopropyl)sulfonamide, i.e. the compound of formula I with the chemical structure depicted hereinafter, is of particular interest. This compound shows pronounced activity against HCV, has an attractive pharmacokinetic profile, and is well-tolerated. This compound can be prepared by the synthesis procedure described in Example 5 of WO 2007/014926.

The RNA-dependent RNA polymerase NS5B is essential for replication of the RNA genome. Both nucleoside and non-nucleoside inhibitors of this enzyme are known.

For example WO 2008/043704 describes a number of nucleoside inhibitors, one of which is 4-amino-1-((2R,3S,4S,5R)-5-azido-4-hydroxy-5-hydroxymethyl-3-methyl-tetrahydrofuran-2-yl)-1H-pyrimidin-2-one, i.e. the compound of formula II with the chemical structure depicted hereinafter. This compound can be prepared by the synthesis procedure described in Example 1 of WO 2008/043704.

WO2010/003658 describes a number of non-nucleoside inhibitors, one of which is the compound of formula III with the chemical structure depicted hereinafter. This compound can be prepared by the synthesis procedure described in Example 1 of WO2010/003658.

### DESCRIPTION OF THE INVENTION

The present invention relates to a combination comprising the compound of formula I:

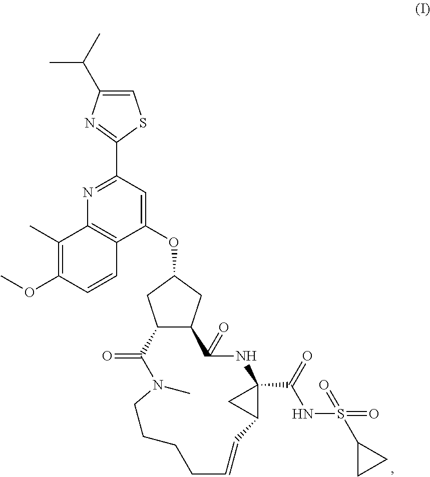 [SMILES: COc1ccc2c(O[C@@H]3C[C@H]4C(=O)N[C@]5(C(=O)NS(=O)(=O)C6CC6)C[C@H]5/C=C/CCCCN(C)C(=O)[C@@H]4C3)cc(-c3nc(C(C)C)cs3)nc2c1C]

or a pharmaceutically acceptable salt thereof,
the compound of formula II:

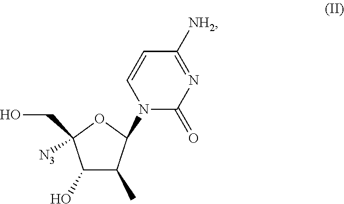 [SMILES: C[C@@H]1[C@H](n2ccc(N)nc2=O)O[C@@](CO)(N=[N+]=[N-])[C@H]1O]

or a pharmaceutically acceptable salt thereof,
and the compound of formula III:

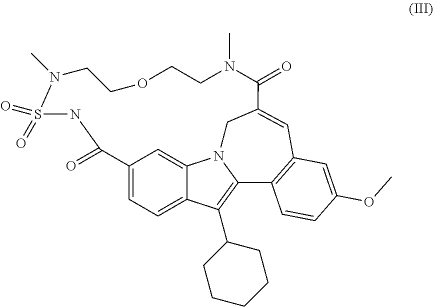 [SMILES: COc1ccc2c(c1)C=C1Cn3c-2c(C2CCCCC2)c2ccc(cc23)C(=O)NS(=O)(=O)N(C)CCOCCN(C)C1=O]

or a pharmaceutically acceptable salt thereof.

It has been found that the combination of these active ingredients increases anti-HCV activity and suppress the emergence of resistant colonies thereby raising the genetic barrier to resistance compared to each single inhibitor alone. It has also been found that the combination of these active ingredient improve clearance of replicon HCV RNA, even in low doses of the three direct antivirals of formula I, II and III.

The compounds of formula I, formula II or formula III may be used in pharmaceutically acceptable salt forms or in free (i.e. non-salt) form. Salt forms can be obtained by treating the free form with an acid or base. Of interest are the pharmaceutically acceptable acid and base addition salts, which are meant to comprise the therapeutically active non-toxic acid and base addition salt forms that the compounds of formula I and II are able to form. The pharmaceutically acceptable acid addition salts of the compounds of formula I and II can conveniently be obtained by treating the free form with such appropriate acid. Appropriate acids comprise, for example, inorganic acids such as hydrohalic acids, such as hydrobromic acid, or in particular hydrochloric acid; or sulfuric, nitric, phosphoric and the like acids; or organic acids such as, for example, acetic, propanoic, hydroxyacetic, lactic, pyruvic, oxalic, malonic, succinic, maleic, fumaric, malic (i.e. hydroxybutanedioic acid), tartaric, citric, methanesulfonic, ethanesulfonic, benzenesulfonic, p-toluenesulfonic, cyclamic, salicylic, p-aminosalicylic, pamoic and the like acids. The compounds of formula I may also be converted into the pharmaceutically acceptable metal or amine addition salt forms by treatment with appropriate organic or inorganic bases. Appropriate base salt forms comprise, for example, the ammonium salts, the alkali and earth alkaline metal salts, e.g. the lithium, sodium or potassium salts; or the magnesium or calcium salts; salts with organic bases, e.g. the benzathine, N-methyl-D-glucamine, hydrabamine salts, and salts with amino acids such as, for example, arginine, lysine, and the like. The term addition salt form is meant to also comprise any solvates that the compounds of formula I or formula II, as well as the salts thereof, may form. Such solvates are, for example, hydrates, alcoholates, e.g. ethanolates, and the like. Of interest are the free (i.e. non-salt) form of the compound of formula II, or the pharmaceutically acceptable salt forms of the compound of formula I.

The EC₅₀ ratio between the active ingredients of formula I, II and III in the combinations of the invention may vary. As used herein the term “EC₅₀ ratio” refers to the ratio of the EC₅₀ value of the compound of formula I to the EC₅₀ value of the compound of formula II, and to the EC₅₀ value of the compound of formula III, said EC₅₀ values being obtained in the HCV replicon test. The latter in particular is the test method described hereinafter. In this test, the average EC₅₀ value of compound I was found to be 8 nM and the average EC₅₀ value of compound II to be 5 μM and the reported EC₅₀ value of compound III in WO2010/003658to be 0.07 μM.

Based on the above EC₅₀ values, effective blood plasma levels can be determined by multiplying the EC₅₀ values with a factor that expresses plasma protein binding and a factor that represents a safety margin. The latter factor can be set at about 10. Protein binding can be determined by measuring the amount bound to blood proteins such as human serum albumin, lipoprotein, glycoprotein, α, β, and γ globulins. Effective blood plasma levels, which can also be referred to as virological active doses, represent those doses that are needed to provide effective anti-viral activity, i.e. doses that effectively reduce viral load. The viral load is effectively reduced when it is reduced about two or more orders of magnitude, preferably below the detection limit of the virus. From the virological active doses, the dose (or amount of drug) to be administered can be calculated with the volume of distribution (V_{D}), which is also known as apparent volume of distribution. This is a pharmacological term used to quantify the distribution of a medication between plasma and the rest of the body after oral or parenteral dosing. It is defined as the volume in which the amount of drug would need to be uniformly distributed to produce the observed blood concentration. The V_{D} can be determined in animal models in which predetermined amounts of the active substance are administered and the blood plasma levels are measured.

The amounts of the compound of formula I in the combinations of the invention that are administered on a daily basis may vary from about 1 mg to about 2500 mg, about 5 mg to about 1000 mg, or from about 10 mg to about 500 mg, or from about 25 mg to about 250 mg, or from about 25 mg to about 200 mg. Examples of daily amounts of the compound of formula I are 25 mg, 50 mg, 75 mg, 100 mg, 125 mg, 150 mg, 200 mg, and 400 mg. The amounts of the compound of formula II that are administered on a daily basis may vary from about 250 mg to about 20,000 mg, or from about 500 mg to about 16,000 mg, or from about 1000 mg to about 12,000 mg, or from about 3000 mg to about 12,000 mg, or from about 3000 mg to about 6000 mg. Examples of daily amounts of the compound of formula II are 3000 mg, 4500 mg, 6000 mg, 12,000 mg. The amounts of the compound of formula III that are administered on a daily basis may vary from about 10 mg to about 2500 mg, or from about 20 mg to about 1000 mg, or from about 50 mg to about 750 mg, or from about 100 mg to about 500 mg, or from about 125 mg to about 250 mg. Examples of daily amounts of the compound of formula III are 100 mg, 150 mg, 200 mg, 500 mg and 1000 mg. All amounts mentioned in this and the following paragraphs refer to the free form (i.e. non-salt form). The above values represent free-form equivalents, i.e. quantities as if the free form would be administered. If salts are administered the amounts need to be calculated in function of the molecular weight ratio between the salt and the free form.

The above mentioned daily doses are calculated for an average body weight of about 70 kg and should be recalculated in case of paediatric applications, or when used with patients with a substantially diverting body weight.

The dosages may be presented as one, two, three or four or more sub-doses administered at appropriate intervals throughout the day. The dosage used preferably corresponds to the daily amount of the compound of formula I, or of the compound of formula II, mentioned above, or a sub-dose thereof, such as ½, ⅓, or ¼ thereof. A dosage form may contain the compound I, the compound II, or the compound III, or all three together, in an amount equal to the ranges or quantities mentioned in the previous paragraphs, for example a dosage form may contain 25 mg, 50 mg, 100 mg, 200 mg of compound I, 250 mg, 500 mg, 1000 mg, 1500 mg, or 2000 mg of compound II, 100 mg, 150 mg, 200 mg, 500 mg or 1000 mg of compound III, either in separate formulations or in a combined formulation. In one embodiment, the compound of formula I is administered once daily (q.d.), in particular as one dose per day, and the compound of formula II is administered once or twice daily (q.d. or b.i.d.), in particular as one or as two doses per day, and the compound of formula III is administered once or twice daily (q.d. or b.i.d.), in particular as one or as two doses per day. In the instance where all three the compounds of formula I and of formula II and of formula III are to be administered once daily, this can be accomplished by administering three separate doses, one with compound I, the other with compound II, and the third with compound III, or by administering a combined dose containing compound I and compound II and compound III.

The combinations of the invention may be administered once, twice, three, four, or if desired multiple times daily. In one embodiment, the combination is administered once daily. In another embodiment, the combination is administered twice daily, or three times per day. Administration of dosages may be by separate dosage forms, i.e. dosage forms only containing compound I or only compound II or only compound III; or by combined dosage forms containing active ingredients I, II and III. Also, a mix of using a combined dosage form and separate dosage forms can be used. Dosage forms that can be administered are described hereinafter, oral dosage forms, in particular tablets or capsules being preferred.

Active ingredients may be formulated in pharmaceutical compositions either separately or as a combined pharmaceutical composition. In the latter instance, there is provided a pharmaceutical composition comprising a therapeutically effective amount of the compound of formula I, or a pharmaceutically acceptable salt thereof, and the compound of formula II, or a pharmaceutically acceptable salt thereof, and the compound of formula III, or a pharmaceutically acceptable salt thereof, the foregoing being as specified herein, and a pharmaceutically acceptable carrier. A therapeutically effective amount in this context is an amount sufficient to act in a prophylactic way against, or to stabilize or to reduce HCV infection, in infected subjects or subjects being at risk of being infected. Therapeutically effective amounts may in particular correspond to the amounts mentioned above for administration on a daily base or of the subdoses thereof in ease of multiple daily administrations.

In a further aspect, this invention relates to a process of preparing a pharmaceutical composition as specified herein, which comprises intimately mixing a pharmaceutically acceptable carrier with a therapeutically effective amount of the compound of formula I, or a pharmaceutically acceptable salt thereof, and a therapeutically effective amount of the compound of formula II, or a pharmaceutically acceptable salt thereof, and a therapeutically effective amount of the compound of formula III, or a pharmaceutically acceptable salt thereof.

The combinations provided herein may also be formulated as a combined preparation for simultaneous, separate or sequential use in HCV therapy. In such a case, the compound of formula I is formulated in a pharmaceutical composition containing other pharmaceutically acceptable excipients, and the compound of formula II is formulated separately in a pharmaceutical composition containing other pharmaceutically acceptable excipients, and the compound of formula III is formulated separately in a pharmaceutical composition containing other pharmaceutically acceptable excipients.

Conveniently, these separate pharmaceutical compositions can be part of a kit for simultaneous, separate or sequential use.

The individual components of the combination of the present invention can be administered simultaneously or separately at different times during the course of therapy or concurrently in divided or single combination forms.

Therefore, the compounds of formula I, II and III, individually or combined, may be formulated into various pharmaceutical compositions suitable for administration purposes. In these, a therapeutically effective amount of the particular compound, or of all three compounds, is combined with a pharmaceutically acceptable carrier, which carrier may take a wide variety of forms depending on the form of preparation desired for administration. Pharmaceutical compositions may be prepared as medicaments to be administered orally, parenterally (including subcutaneously, intramuscularly, and intravenously), rectally, transdermally, bucally, or nasally. Suitable compositions for oral administration include powders, granulates, aggregates, tablets, compressed or coated pills, dragees, sachets, hard or gelatin capsules, syrups and suspensions. Suitable compositions for parenteral administration include aqueous or non-aqueous solutions or emulsions, while for rectal administration suitable compositions for administration include suppositories with a hydrophilic or hydrophobic vehicle. For topical administration there can be used suitable transdermal delivery systems and for nasal delivery there can be used suitable aerosol delivery systems.

For example, in preparing the compositions for oral administration, any of the usual pharmaceutical media may be employed such as, for example, water, glycols, oils, alcohols and the like in the case of oral liquid compositions such as suspensions, syrups, elixirs, emulsions and solutions; or solid carriers such as starches, sugars, kaolin, lubricants, binders, disintegrating agents and the like in the case of solid compositions. For parenteral compositions, the carrier will usually comprise sterile water, at least in large part, though other ingredients, such as solubilizers, emulsifiers or further auxiliaries may be added thereto. Injectable solutions may be prepared in which the carrier comprises saline solution, glucose solution or a mixture of both. Injectable suspensions may also be prepared in which case appropriate liquid carriers, suspending agents and the like may be employed. Also included are solid form preparations intended to be converted, shortly before use, to liquid form preparations such as powders for reconstitution. In the compositions suitable for percutaneous administration, the carrier optionally comprises a skin penetration enhancing agent and/or a wetting agent, optionally combined with suitable skin-compatible additives in minor proportions. The compounds of formula I or II, or combinations thereof, may also be administered via oral inhalation or insufflation by formulations suited for this type of administration such as a solution, a suspension or a dry powder. Suitable pharmaceutical compositions for administration in the form of aerosols or sprays are, for example, suspensions of the compound of formula I or II, or both, in a pharmaceutically acceptable liquid carrier, such as ethanol or water, or a mixture thereof. If required, the formulation can also additionally contain other pharmaceutical auxiliaries such as surfactants, emulsifiers and stabilizers as well as a propellant. Such a preparation customarily contains the active compound in a concentration from approximately 0.1 to 50%, in particular from approximately 0.3 to 3% by weight.

The pharmaceutical compositions may contain the active ingredient of formula I, or of formula II, or of formula III, or all three combined, in a concentration of about 0.1% to about 50%, or about 1% to about 30%, or about 3% to about 20%, or about 5% to about 20%, all percentages being by weight. In the compositions containing all three the compound formula I, and of formula II and of formula III, the compound of formula I is present in a concentration of about 0.1% to about 50%, or about 1% to about 30%, or about 3% to about 20%, or about 5% to about 20%; and the compound of formula II is present in a concentration of about 3% to about 50%, or about 5% to about 50%, or about 10% to about 50%, or about 10% to about 50%, or about 10% to about 30%; the compound of formula III is present in a concentration of about 0.1% to about 50%, or about 1% to about 30%, or about 3% to about 20%, or about 5% to about 20%.

The pharmaceutical compositions may be conveniently presented in unit dosage form for ease of administration and uniformity of dosage. Examples include tablets (including scored or coated tablets), capsules, pills, suppositories, powder packets, wafers, injectable solutions or suspensions and the like, and segregated multiples thereof. Of interest are solid dosage forms for oral administration such as tablets on capsules.

The solid dosage forms in unit dose form may be packed in any known package, blister packs being preferred, in particular for tablets and capsules. Where the compound of formula I, of formula II and of formula III are formulated separately, they could be packed in separate blisters, but one blister could as well comprise unit dose forms of the compound I as of the compound II as of the compound III, for example one row with units of compound I and another with compound II, and another with compound III. Other possibilities may be possible as well.

The combinations of this invention may be used to treat HCV infections as well as diseases associated with HCV. The diseases associated with HCV include progressive liver fibrosis, inflammation and necrosis leading to cirrhosis, end-stage liver disease, and HCC (hepatocellular carcinoma).

The in vitro antiviral activity against HCV of the compound of formula I or of formula II or of formula III can be tested in a cellular HCV replicon system based on Lohmann et al. (1999) Science 285:110-113, with the further modifications described by Krieger et al. (2001) Journal of Virology 75: 4614-4624 (incorporated herein by reference), which is further exemplified in the examples section. This model, while not a complete infection model for HCV, is widely accepted as the most robust and efficient model of autonomous HCV RNA replication currently available. The in vitro antiviral activity against HCV can also be tested by enzymatic tests.

The combination of the compound of formula I, formula II and the compound of formula III, as specified herein, is useful in the treatment of warm-blooded animals, in particular humans, infected with HCV, and for the prophylaxis of HCV infections.

The present invention therefore furthermore relates to a method of treating a warm-blooded animal, in particular a human, infected by HCV, or being at risk of infection by HCV, said method comprising the administration of an anti-HCV effective amount of a combination of the compound of formula I, of formula II and the compound of formula III, as specified herein. The present invention provides as well a method of treating HCV-related conditions or preventing HCV-related conditions in a mammal comprising administering an anti-virally effective amount of a combination of the compound of formula I, of formula II and the compound of formula II, of formula III, as specified herein.

The combinations of the present invention may be used as medicaments. The present invention also relates to the use of a combination, as described herein, for the manufacture of a medicament for the treatment or the prevention of HCV infection or HCV related conditions.

In a further aspect, the invention relates to a product containing the compound of formula I, formula II and the compound of formula III, and optionally another anti-HCV compound, as a combined preparation for simultaneous, separate or sequential use in the treatment of HCV infections.

The combinations of the present invention in turn may be combined with one or more further anti-HCV compounds. Of interest are combinations with IFN-α (pegylated or not) and/or ribavirin.

The other agents that may be co-administered with the combinations of the present invention may be administered as separate formulations or may be co-formulated with one or more of the active ingredients of formula I, of formula II or of formula III.

The combinations of the present invention, including those with other anti-HCV agents, may also be combined with an agent that has a positive effect on drug metabolism and/or pharmacokinetics that improve bioavailabilty, e.g. ritonavir or a pharmaceutically acceptable salt thereof. The ritonavir may be used as separate formulation, or may be co-formulated with one or more of the active agents of the combinations of the present invention. The weight/weight ratio of the compound of formula I or of the compound of formula II or of the compound of formula III to ritonavir may be in the range of from about 10:1 to about 1:10, or from about 6:1 to about 1:6, or from about 1:1 to about 10:1, or from about 1:1 to about 6:1, or from about 1:1 to about 4:1, or from about 1:1 to about 3:1, or from about 1:1 to about 2:1.

In still a further aspect of the invention, there are provided combinations of the compound of formula (I), the compound of formula III and ester pro-drugs of the compound of formula II. These comprise compounds of formula II described in WO 2008/043704, in particular the 4′ and 5′ hydroxy esters, which can be represented by formula IIa:

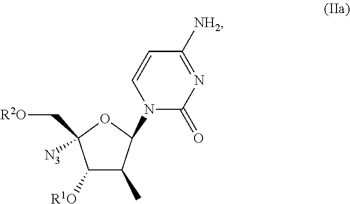

or a pharmaceutically acceptable salt thereof, wherein R¹ is hydrogen and R² is C₁₋₁₈alkyl-CO—; or R² is hydrogen and R¹ is C₁₋₁₈alkyl-CO—; or both R¹ and R² are C₁₋₁₈alkyl-CO—; wherein each C₁₋₁₈alkyl independently is an unbranched or branched saturated hydrocarbon group having from one to 18 carbon atoms; and wherein each C₁₋₁₈alkyl in particular is C₁₋₆alkyl and more in particular is C₃₋₄alkyl. Examples of such ester prodrugs are compounds of formula IIa wherein R¹ is hydrogen and R² is isopropyl; or wherein R² is hydrogen and R¹ is isopropyl-CO—; or wherein both R¹ and R² are isopropyl-CO—. The term isopropyl-CO— refers to an ester of isobutyric acid, which can also be referred to as isobutyryl. Pharmaceutically acceptable salts of the prodrugs of formula IIa are as described above for the salts of the compound of formula II.

In this aspect, the compound of formula (II) is replaced by an equivalent amount of an ester prodrug in the combinations, formulations, uses, or methods described above.

As used herein, the term “about” has its conventional meaning In particular embodiments, when in relation to a numerical value, it may be interpreted to mean the numerical value ±10%, or ±5%, or ±2%, or ±1%, or ±0.5%, or ±0.1%. In other embodiments, the precise value is meant, i.e. by leaving out the word “about”.

### FIGURES

FIG. 1. Effect of combining (A) compound I and compound II, (B) compound I and compound III, and (C) compound II and compound III on antiviral activity. Three-dimensional synergy plots at the 95% confidence interval (CI), as produced by the MacSynergy™ II software for representative experiments are shown.

FIG. 2. Cell colony formation in the presence of compounds I, II and III alone (A), or in combination (B and C). The number of surviving cell colonies is indicated on the right lower corner for each cell culture dish. EC₅₀, means 50% effective concentration.

FIG. 3. Clearance of HCV RNA from replicon-containing cells in the presence of compounds I, II and III alone and in combination. The rebound phase is shaded in grey, and the RT-PCR cut-off is shown as a red line. The number of surviving replicon cell colonies is indicated. HCV, hepatitis C virus; RNA, ribonucleic acid; RT-PCR, reverse transcription polymerase chain reaction.

### EXAMPLES

The following examples are intended to illustrate the present invention and not to limit it thereto.

### Example 1

### Activity of the Compounds of Formula I, II and III

### Replicon Assay

The compound of formula I, II and III were examined for activity in the inhibition of HCV RNA replication in a cellular assay. The cellular assay was based on a bicistronic expression construct, as described by Lohmann et al. (1999) Science vol. 285 pp. 110-113 with modifications described by Krieger et al. (2001) Journal of Virology 75: 4614-4624, in a multi-target screening strategy. In essence, the method was as follows.

The assay was based on the stably transfected cell line Huh-7 luc/neo (hereafter referred to as Huh-Luc). This cell line harbors an RNA encoding a bicistronic expression construct comprising the wild type NS3-NS5B regions of HCV type 1b translated from an Internal Ribosome Entry Site (IRES) from encephalomyocarditis virus (EMCV), preceded by a reporter portion (FfL-luciferase), and a selectable marker portion (neo^{R}, neomycine phosphotransferase). The construct is bordered by 5′ and 3′ NTRs (non-translated regions) from HCV type 1b. Continued culture of the replicon cells in the presence of G418 (neo^{R}) is dependent on the replication of the HCV RNA. The stably transfected replicon cells that express HCV RNA, which replicates autonomously and to high levels, encoding inter alia luciferase, are used for screening the antiviral compounds.

The replicon cells were plated in 384 well plates in the presence of the test and control compounds which were added in various concentrations. Following an incubation of three days, HCV replication was measured by assaying luciferase activity (using standard luciferase assay substrates and reagents, and a Perkin Elmer ViewLux™ ultraHTS microplate imager). Replicon cells in the control cultures have high luciferase expression in the absence of any inhibitor. The inhibitory activity of the compound was monitored on the Huh-Luc cells, enabling a dose-response curve for each test compound. EC₅₀ values were then calculated, which value represents the amount of the compound required to decrease by 50% the level of detected luciferase activity, or more specifically, the ability of the genetically linked HCV replicon RNA to replicate.

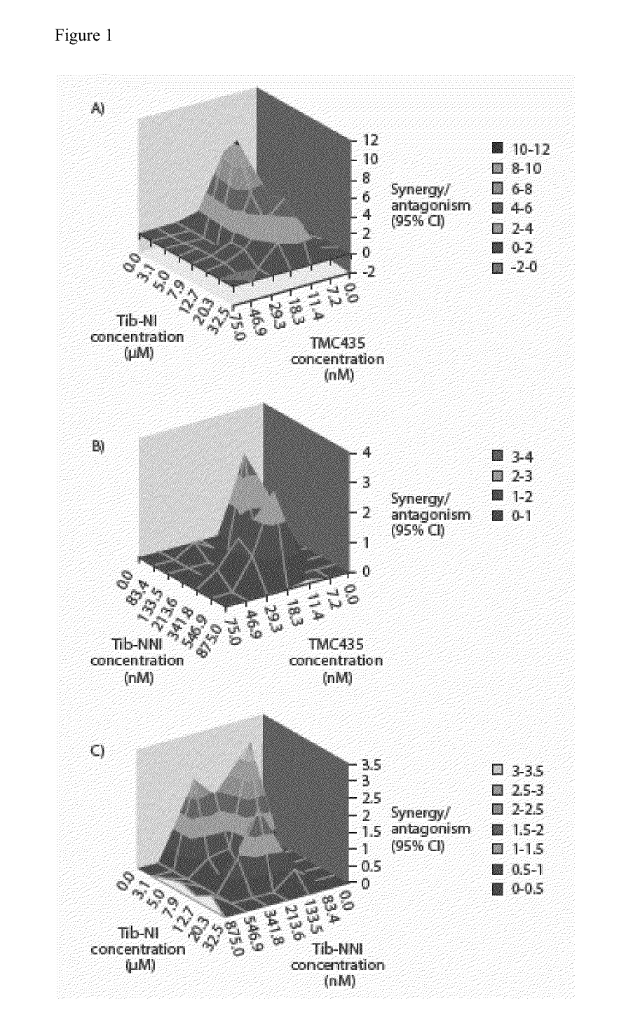

The effect of combining compounds I, II and III on anti-HCV activity is shown in FIG. 1 and Table 1

TABLE 1
Antiviral activity of different combinations of
TMC435, Tib-NNI and Tib-NI.

| | Synergy | Antagonism | |
|---|---|---|---|
| | volumes | volumes | |
| | at 95% CI | at 95% CI | |
| Combinations | (μM² %) | (μM² %) | Combination effect |
| TMC435 + Tib-NNI | 5.67 | −0.43 | Additive |
| | | | (insignificant |
| | | | synergism) |
| TMC435 + Tib-NI | 37.89 | 0.11 | Synergistic |
| Tib-NNI + Tib-NI | 16.91 | −0.61 | Additive |
| | | | (insignificant |
| | | | synergism) |

Synergy and antagonism volumes at the 95% confidence interval (CI), as produced by the MacSynergy™ II software. Synergy volumes of <25, 25-50, 50-100 and >100 indicate insignificant synergism, slight synergism, moderate synergism and strong synergism, respectively. Results shown are averages from two or more experiments

Treatment of the cells with compound I in combination with compound III or compound II resulted in additive or synergistic anti-HCV activity, respectively. Treatment with compound III in combination with compound II resulted in additive anti-HCV activity.

No cytotoxicity was observed with any of the combinations tested.

### Example 2

### Colony Formation

Colony formation was determined using HCV-genotype-1b-replicon containing cells in the presence of the compounds of formula I, II and III. Huh7-Luc replicon cells (20,000) were seeded in a 10 cm dish containing DMEM plus 10% FCS and treated with different concentrations of a single inhibitor or with two inhibitors combined, in the presence of 1,000 μg/mL G418. Cells were incubated, and inhibitor and media were refreshed twice weekly. When significant cell death had occurred (approximately 2-3 weeks), the remaining colonies were stained with neutral red and counted.

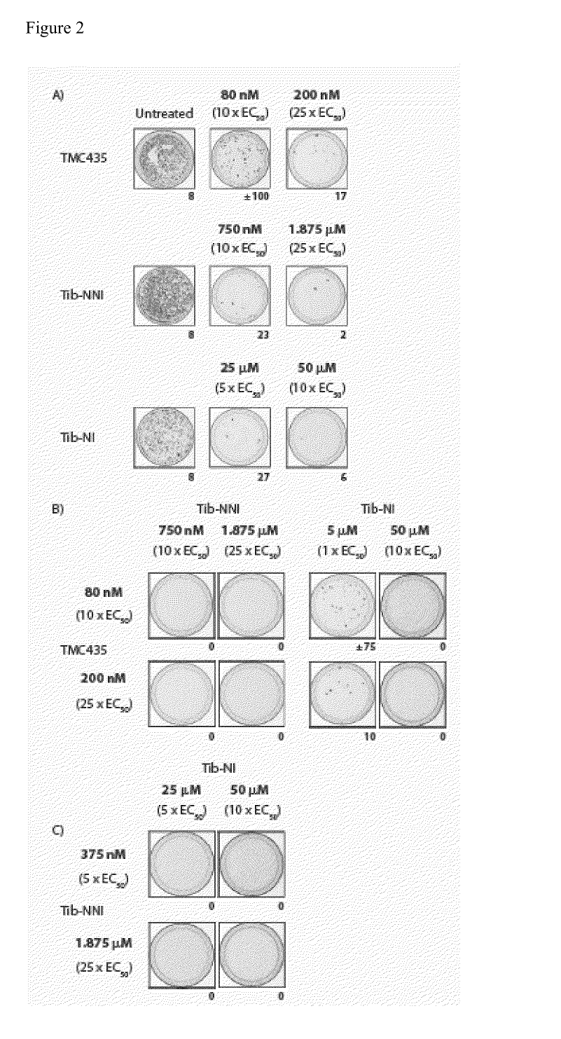

Cell colony formation, in the presence of compounds I, II and III alone and in combination, is shown in FIG. 2.

Increasing concentrations of each inhibitor alone resulted in a dose-dependent reduction in colony formation but did not completely prevent resistant replicon colony formation. Treatment with compound I in combination with compound III or compound II prevented the formation of resistant replicon colonies. Treatment with compound III in combination with compound II prevented the formation of resistant replicon colonies at the lowest concentration tested.

### Example 3

### Replicon Clearance-Rebound Assay

HCV-replicon ribonucleic acid (RNA) levels during clearance-rebound were assessed using HCV-genotype-1b-replicon-containing cells. Huh7-Luc replicon cells (300,000) were seeded in a 10 cm dish containing DMEM plus 10% FCS and cultured in the presence of one or more of the inhibitors in the absence of G418 (clearance phase). Cells were passaged as needed (typically twice weekly) and HCV RNA was extracted. After 14 days, inhibitors were removed and cells were incubated for 21 days in the presence of 250 μg/mL G418 (rebound phase). HCV replicon RNA and cellular RPL13A transcript levels were quantified using real-time quantitative reverse transcription polymerase chain reaction (qRT-PCR), and HCV replicon RNA levels were normalised to RPL13A transcript levels. The number of cell colonies observed at the end of the experiment was counted.

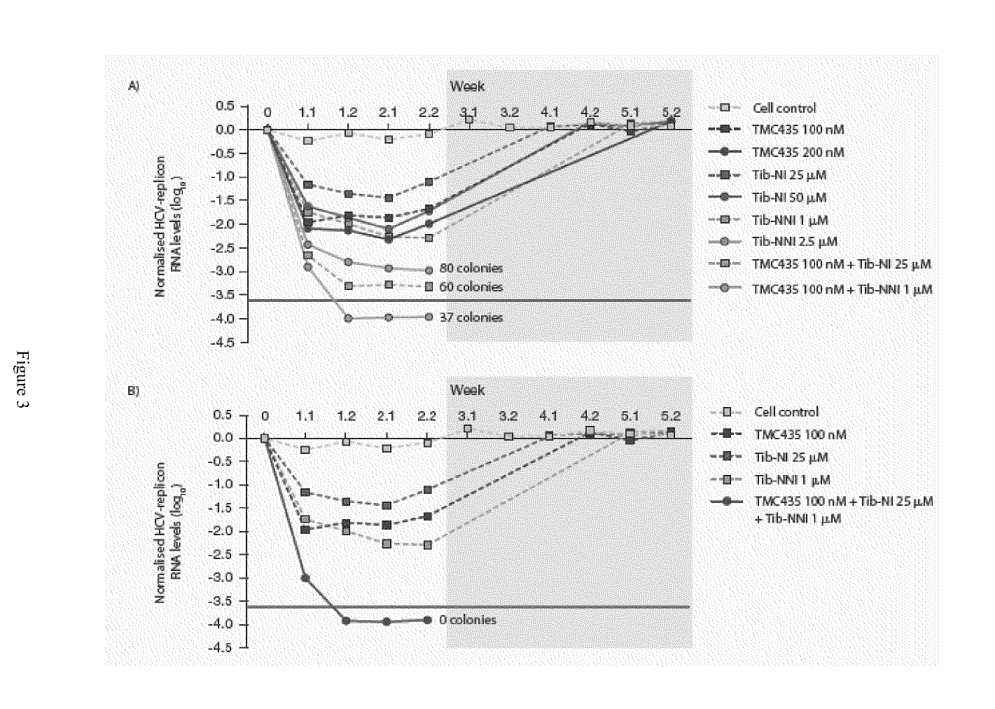

Clearance of HCV RNA from replicon-containing cells in the presence (clearance phase) and absence (rebound phase) of compounds I, II or III alone and in combination are shown in FIG. 3.

All three inhibitors reduced replicon HCV RNA levels during the 2-week clearance phase, but did not lead to total clearance of replicon HCV RNA from cells. Treatment with compound I in combination with compound III or compound II increased the initial HCV RNA reduction, although a few replicon colonies were observed in the rebound phase, suggesting incomplete replicon HCV RNA clearance for some combinations. Treatment with a combination of all three inhibitors at the lowest concentrations tested resulted in a pronounced reduction in replicon HCV RNA and the most efficient replicon clearance.

1. A combination comprising (i) the compound of formula I: 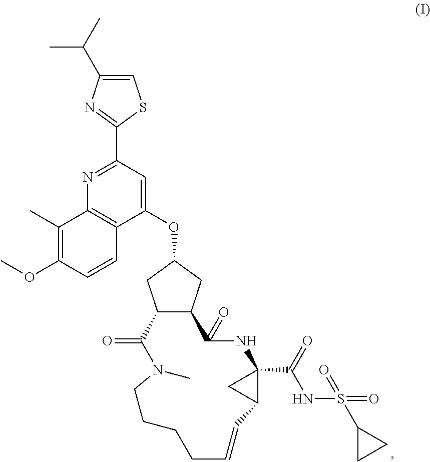 [SMILES: COc1ccc2c(O[C@@H]3C[C@H]4C(=O)N[C@]5(C(=O)NS(=O)(=O)C6CC6)C[C@H]5/C=C/CCCCN(C)C(=O)[C@@H]4C3)cc(-c3nc(C(C)C)cs3)nc2c1C]
or a pharmaceutically acceptable salt thereof,
and (ii) the compound of formula III: 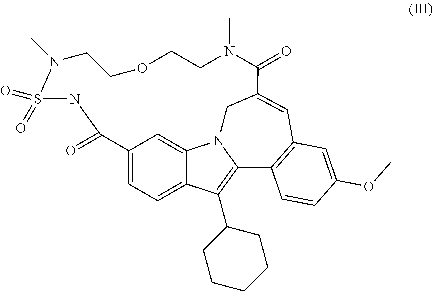 [SMILES: COc1ccc2c(c1)C=C1Cn3c-2c(C2CCCCC2)c2ccc(cc23)C(=O)NS(=O)(=O)N(C)CCOCCN(C)C1=O]
or a pharmaceutically acceptable salt thereof.

2. The combination of claim 1, wherein the compounds of formula I and III are formulated as a combined pharmaceutical composition.

3. The combination of claim 1, wherein the compounds of formula I and III are formulated separately.

4. The combination of claim 1, further comprising ritonavir or a pharmaceutically acceptable salt thereof.

5. The combination as claimed in claim 4, wherein ritonavir is co-formulated with one or more of the active agents of the combinations.

6. The combination as claimed in claim 4, wherein ritonavir is used as a separate formulation.

7. The combination of claim 1, combined with a further agent selected from ribavirin and interferon.

8. A pharmaceutical composition comprising a combination as claimed in claim 1, and a pharmaceutically acceptable carrier.

9. A process for the preparation of the pharmaceutical composition of claim 8 comprising intimately mixing a pharmaceutically acceptable carrier with a therapeutically effective amount of the compound of formula I, or a pharmaceutically acceptable salt thereof, and a therapeutically effective amount of the compound of formula III, or a pharmaceutically acceptable salt thereof.

10. A method for treating an HCV infection in a patient comprising administering to said patient the compound of formula I: 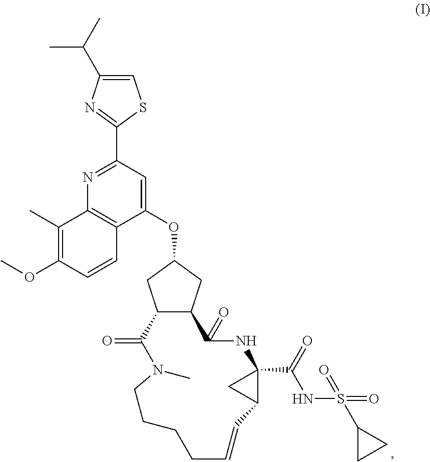 [SMILES: COc1ccc2c(O[C@@H]3C[C@H]4C(=O)N[C@]5(C(=O)NS(=O)(=O)C6CC6)C[C@H]5/C=C/CCCCN(C)C(=O)[C@@H]4C3)cc(-c3nc(C(C)C)cs3)nc2c1C]
or a pharmaceutically acceptable salt thereof,
and (ii) the compound of formula III: 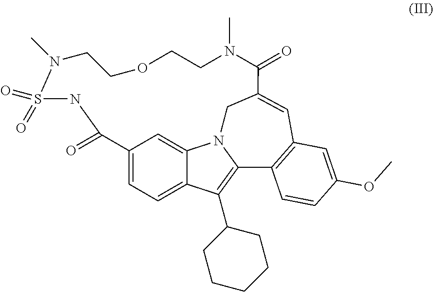 [SMILES: COc1ccc2c(c1)C=C1Cn3c-2c(C2CCCCC2)c2ccc(cc23)C(=O)NS(=O)(=O)N(C)CCOCCN(C)C1=O]
or a pharmaceutically acceptable salt thereof.

11. The method of claim 10, further comprising administering ritonavir to said patient.

12. The method of claim 10, further comprising administering ribavirin to said patient.

13. The method of claim 10, further comprising administering interferon to said patient.

14. The method of claim 10, wherein said compound I or pharmaceutically acceptable salt thereof and said compound III or pharmaceutically acceptable salt thereof are administered simultaneously to said patient.

15. The method of claim 10, wherein said compound I or pharmaceutically acceptable salt thereof and said compound III or pharmaceutically acceptable salt thereof are administered separately to said patient.

16. The method of claim 15, wherein said compound I or pharmaceutically acceptable salt thereof and said compound III or pharmaceutically acceptable salt thereof are administered sequentially to said patient.
